(19) United States
(12) Patent Application Publication
(10) Pub. No.: US 2025/0213831 A1
(43) Pub. Date: Jul. 3, 2025

(54) VENTRICULAR CATHETERS AND OTHER EMBODIMENTS

(71) Applicant: Wayne State University, Detroit, MI (US)

(72) Inventors: Carolyn Harris, Detroit, MI (US); Ahmad Faryami, Detroit, MI (US); Adam Menkara, Detroit, MI (US); Daniel Viar, Detroit, MI (US)

(73) Assignee: Wayne State University, Detroit, MI (US)

(21) Appl. No.: 18/690,227

(22) PCT Filed: Sep. 9, 2022
(86) PCT No.: PCT/US2022/076236

§ 371 Date: Mar. 7, 2024

Related U.S. Application Data
(63) Continuation-in-part of application No. PCT/US2022/076230, filed on Sep. 9, 2022.
(60) Provisional application No. 63/242,468, filed on Sep. 9, 2021.

(30) Foreign Application Priority Data
Aug. 19, 2021 (CN) 202110951708.5

(51) Int. Cl. A61M 27/00

(52) U.S. Cl. CPC A61M 27/006; A61M 2202/0413

(57) ABSTRACT

Ventricular catheters and other embodiments are provided. The catheter for shunting fluid includes a tip end, a first portion, and a second portion. The first portion is between and in fluid communication with the tip end and the second portion. The first portion is configured to have a tapered shape. The first portion includes multiple rows of holes.

### CROSS-REFERENCE TO RELATED APPLICATIONS

This application is the 371 National Phase of International Application No. PCT/US22/76236, filed on Sep. 9, 2022, which claims priority to PCT Application No. PCT/US2022/076230, filed Sep. 9, 2022; and priority to and the benefit of the earlier filing of U.S. Provisional Application No. 63/242,468, filed on Sep. 9, 2021. Each of these applications is incorporated by reference herein in its entirety.

### STATEMENT REGARDING FEDERALLY SPONSORED RESEARCH OR DEVELOPMENT

This invention was made with government support under Grant No. 5R01NS094570-05 awarded by the National Institutes of Health (National Institute of Neurological Disorders and Stroke; NINDS). The government has certain rights in the invention.

### FIELD OF THE DISCLOSURE

This disclosure relates to catheters for shunting flow of biological fluids in organs or tissues including in vivo or in vitro such as in the context of cerebrospinal fluid in brain tissue, and blood in the circulatory system. It further relates to improvements in systems, system elements, and methods related to regulating fluid flow and circulation of fluids using such catheters.

### BACKGROUND OF THE DISCLOSURE

Hydrocephalus, an imbalance between cerebrospinal fluid (CSF) production and absorption, is diagnosed in more than 1 in 500 people in the United States. Approximately 80% of these patients will suffer long-term neurological deficits. Genetic diseases, meningitis, subarachnoid hemorrhage, stroke, traumatic brain injury, or tumors, cause hydrocephalus.

The common treatment for all hydrocephalus patients is CSF drainage by shunting. Despite all efforts to date, shunts still have the highest failure rate of any neurological device. A shocking 98% of shunts fail after just ten years, a rate bumped up by the 80% of patients who suffer from tens if not hundreds of repetitive shunt failures. Shunts fail after becoming obstructed with attaching glia, creating a substrate for more glia or other cells and tissues (e.g. choroid plexus) to secondarily bind and block the flow of CSF through the shunt. For additional discussion, see U.S. Patent Publication No. 2012/0060622 (Harris et al.).

Hydrocephalus patients can have a diminished quality of life and suffer from long-term neurologic deficits because of the failure of current treatments in the field, most of which involve diversion of cerebrospinal fluid (CSF) with shunts. Despite our efforts for nearly seven decades, shunts still have the highest failure rate of any neurological device: 98% of all shunts fail after ten years. This failure rate is the dominant contributor to the $2 billion-per-year cost that hydrocephalus incurs on our health care system.

While many factors such as infection and disconnection could lead to shunt obstruction and eventual failure, the statistics tell us that most shunts fail by becoming blocked with cells and tissues. But how does this happen? The literature predicts that there are four mechanisms: (1) cells coming from the brain, attaching and blocking the ventricular catheter; (2) cells, protein, and debris from the CSF attaching and blocking the ventricular catheter; (3) blockage by the ventricular catheter laying on the ventricular wall's epithelial cells; (4) blockage by the choroid plexus (lined with epithelial cells).

There is an ongoing urgent need to improve hydrocephalus treatment. Shunts are far from ideal even when they are not occluded, and patients often experience discomfort such as headaches and pain on a regular basis which directly impact their quality of life. Catheters as necessary components of the shunt system can be improved to reduce the shunt failure rate.

### SUMMARY OF THE DISCLOSURE

This Summary is provided to introduce a selection of concepts in a simplified form that is further described below in the Detailed Description. This Summary is not intended to identify all key features or essential features of the claimed subject matter, nor is it intended to be used alone as an aid in determining the scope of the claimed subject matter.

The present disclosure provides a catheter for shunting fluid including a tip end, a first portion, and a second portion. The first portion is between and in fluid communication with the tip end and the second portion. The first portion is configured to have a tapered shape. The first portion includes multiple rows of holes.

In some examples, the tip end is a closed end.

In some examples, the first portion has a first diameter close to the tip end and a second diameter close to the second portion. The first diameter is smaller than the second diameter.

In some examples, the second portion has a constant diameter. In some examples, the second portion has a second diameter.

In some examples, an individual row of the multiple rows of holes includes multiple holes. In some examples, the multiple holes are placed at constant intervals. In some examples, the multiple holes are placed at various intervals. In some examples, the multiple holes have the same diameter. In some examples, the multiple holes have different diameters.

In some examples, an individual row of the multiple rows of holes includes multiple holes, the multiple holes are configured to facilitate a relatively uniform flow rate distribution of the fluid flowing therethrough.

The present disclosure provides a system for shunting fluid, including a ventricular, a distal catheter; and a valve connected between the ventricular catheter and the distal catheter. The ventricular catheter includes a tip end, a first portion, and a second portion. In some examples, the first portion is between and in fluid communication with the tip end and the second portion. The first portion is configured to have a tapered shape. The first portion includes multiple rows of holes.

In some examples, the tip end is a closed end.

In some examples, the first portion has a first diameter close to the tip end and a second diameter close to the second portion. The first diameter is smaller than the second diameter.

In some examples, the second portion has a constant diameter. In some examples, the second portion has a second diameter.

In some examples, an individual row of the multiple rows of holes includes multiple holes. In some examples, the multiple holes are placed at constant intervals. In some examples, the multiple holes are placed at various intervals. In some examples, the multiple holes have the same diameter. In some examples, the multiple holes have different diameters.

In some examples, an individual row of the multiple rows of holes includes multiple holes, the multiple holes are configured to facilitate a relatively uniform flow rate distribution of the fluid flowing therethrough.

The present disclosure provides a ventricular catheter essentially as described or illustrated herein.

The present disclosure provides a ventricular catheter, which is optimized to reduce astrocyte activation. In some examples, the optimization includes at least one of shear reduction or flow redistribution.

The present disclosure provides a ventricular catheter, formatted for use with a hydrocephalus shunt.

The present disclosure provides a use of the ventricular catheter in vitro to study fluid flow, or in vivo to treat or ameliorate symptoms of hydrocephalus.

### BRIEF DESCRIPTION OF DRAWINGS

The detailed description is set forth with reference to the accompanying figures. In the figures, the left-most digit(s) of a reference number identifies the figure in which the reference number first appears. The use of the same reference numbers in different figures indicates similar or identical items or features.

FIG. 1 illustrates pictures of a catheter and blocked catheter holes.

FIG. 2 illustrates an example conventional catheter.

FIG. 3A, FIG. 3B, and FIG. 3C illustrate an example catheter in accordance with this disclosure.

FIG. 4 illustrates a horizontal head computed tomography (CT) image showing a catheter tip implanted in a patient's brain.

FIG. 5 illustrates an example shunt system in accordance with implementations of this disclosure.

FIG. 6A shows a simulation result of the hydrodynamics of flow through a catheter in accordance with implementations of this disclosure.

FIG. 6B illustrates a simulation result of the pressure contours inside the catheter in accordance with implementations of this disclosure.

FIG. 6C illustrates a simulation result of the velocity contours inside the catheter in accordance with implementations of this disclosure.

### DETAILED DESCRIPTION

Hydrocephalus, an imbalance between cerebrospinal fluid production and absorption, is diagnosed in more than 1 in 500 people in the United States. Approximately 80% of these patients will suffer long-term neurological deficits. Genetic diseases, meningitis, subarachnoid hemorrhage, stroke, traumatic brain injury, or tumors cause hydrocephalus. The common treatment for all hydrocephalus patients is CSF drainage by shunting.

Despite all prior efforts, shunts still have the highest failure rate of any neurological device. A shocking 98% of shunts fail after just ten years, a rate bumped up by the 80% of patients who suffer from tens if not hundreds of repetitive shunt failures. Shunts fail after becoming obstructed with attaching glia, creating a substrate for more glia or other cells and tissues (e.g. choroid plexus) to secondarily bind and block the flow of CSF through the shunt.

It has been hypothesized that physiological shear forces acting on medical devices implanted in the brain significantly accelerate the rate of device failure in patients with chronically indwelling neuroprosthetics. In hydrocephalus shunt devices, shear forces arise from cerebrospinal fluid flow. The shunt's unacceptably high failure rate is mostly due to obstruction with adherent inflammatory cells. Astrocytes are the dominant cell type bound directly to obstructing shunts, rapidly manipulating their activation via shear stress-dependent cytokine secretion.

Aspects of the current disclosure are now described in additional detail, as follows: (I) Structure of the Catheter; (II) Shunt Systems Comprising the Catheter; (III) Kits; (IV) Example(s); (V) Example clauses; (VI) References; and (VII) Closing paragraphs.

### (I) Structure of the Catheter

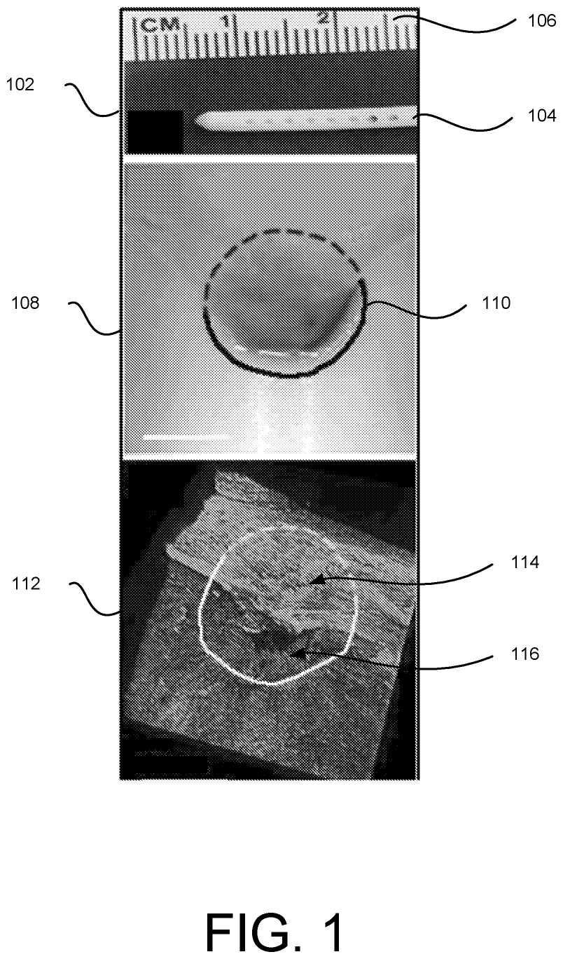

CSF shunt implantation is the most common treatment option for hydrocephalus, yet shunts are plagued by high failure rates: 40% in the first year, and 90% in the first 10 years. Hydrocephalus treatment fails most often because the outflow pathway created by the holes in the shunt's ventricular catheter becomes obstructed with tissue. Up until a few years ago, the most significant studies on shunt failure revealed that shunts most commonly harbor inflammatory glia, lymphocytic inflammation, and foreign body giant cells. Our work shows that the tissue occluding shunts is predominately composed of astrocytes and macrophages, has only sparse microglia, has more activated cells on obstructed shunts than unobstructed, stain positive for proliferative markers, has reactivity that follows the flow, and predominately obstructs shunts as large tissue masses. FIG. 1 illustrates pictures of a catheter and blocked catheter holes. In FIG. 1, picture 102 shows an example catheter 104 along with a ruler 106. Picture 108 shows a hole 110 of the catheter being blocked. The scale bar=500 microns. Picture 112 shows that astrocytes 114 and macrophages 116 block the catheter hole as a large tissue mass.

Recent long-term in vivo data collected in our lab indicate that inflammatory astrocytes are ubiquitous on all shunts. Inflammatory astrocytes make up more than 21% of cells bound to obstructed shunts, and of the occluded masses blocking ventricular catheter holes, a vast majority of the cells are astrocytes, and their number and reactivity peak on failed shunts. Data suggests that inflammatory astrocyte activation on shunts is correlated to a change in flow rate through the shunt holes and indirectly, the shear rate through these holes (Harris et al., Childs Nerv. Syst., 2011; doi.org/10.1007/s00381-011-1430-0; Harris et al., Exp. Neurol. 222:204-210, 2010; Harris et al., J. Biomed. Mater. Res. A 97:433-440, 2011; Harris et al., Fluids Barriers CNS. 2015, doi.org/10.1186/s12987-015-0023-9). Astrocytes have an increased attachment propensity in vitro with increasing flow-induced shear stress. Astrocyte markers have been observed in obstructive masses to be co-localized with proliferative markers, indicating that astrocytes are active on the shunt surface: they produce inflammatory cytokine IL-6 and proliferate.

Most of the CSF volume flows through the proximal holes of the shunt's ventricular catheter, i.e., holes located furthest from the tip of the shunt with less resistance to flow. Computational fluid dynamics simulations have shown that in CSF shunts, the wall shear stress at the proximal holes is greater than 0.5 dyne/cm² (Harris et al., Childs Nerv. Syst., 2011; doi.org/10.1007/s00381-011-1430-0; Lin et al., J. Neurosurg. 99:426-431, 2003; Lee et al., J. R. Soc. Interface 17, 20190884, 2020). This fact increases the shear stress at the proximal segment and is a key driver of a dense glial scar formation around devices causing failure via obstruction (Lin et al., J. Neurosurg. 99:426-431, 2003; Giménez et al., Philos. Trans. A Math. Phys. Eng. Sci. 375:20160294, 2017; Marimuthu et al., Anal. Biochem. 437:161-163, 2013).

CSF shunts removed for obstruction show occlusions to occur most often at the proximal holes (holes located furthest from the tip) with the highest flow (Kestle et al., Pediatr. Neurosurg. 33:230-236, 2000). These observations led to a suggestion that shunt geometry with a more uniform flow rate distribution among the shunt's inlet holes would reduce the obstruction occurring at the critical proximal inlet holes, thereby reducing shunt failure rates. As an example of an existing shunt catheter, the Rivulet® (Medtronic Neurosurgery) shunt was developed with a design consisting of decreasing hole diameters from the distal to proximal end (Lin et al., J. Neurosurg. 99:426-431, 2003). However, shear stress will be higher in the proximal holes of these shunts. Based on the fluid shear stress equation of τ=μ du/dy, where μ is dynamic viscosity, and du/dy is the gradient of velocity in the direction perpendicular to the flow, since the gradient velocity of the decreasing hole diameters is higher, shear stress will be higher for the proximal holes. Based on our hypothesis and other reports of the correlation between increased shear stress along the shunt/CSF interface to result in increased occlusion (Harris et al., Childs Nerv. Syst., 2011; doi.org/10.1007/s00381-011-1430-0; Harris et al., Exp. Neurol. 222:204-210, 2010; Harris et al., J. Biomed. Mater. Res. A 97:433-440, 2011; Galarza et al., Child' Nerv. Syst. 34:267-276, 2018; Weisenberg et al., J. Neurosurg. 2017, doi.org/10.3171/2017.5.JNS161882), it may be necessary to improve shunt design to decrease shear stress through all its holes or at best the proximal holes.

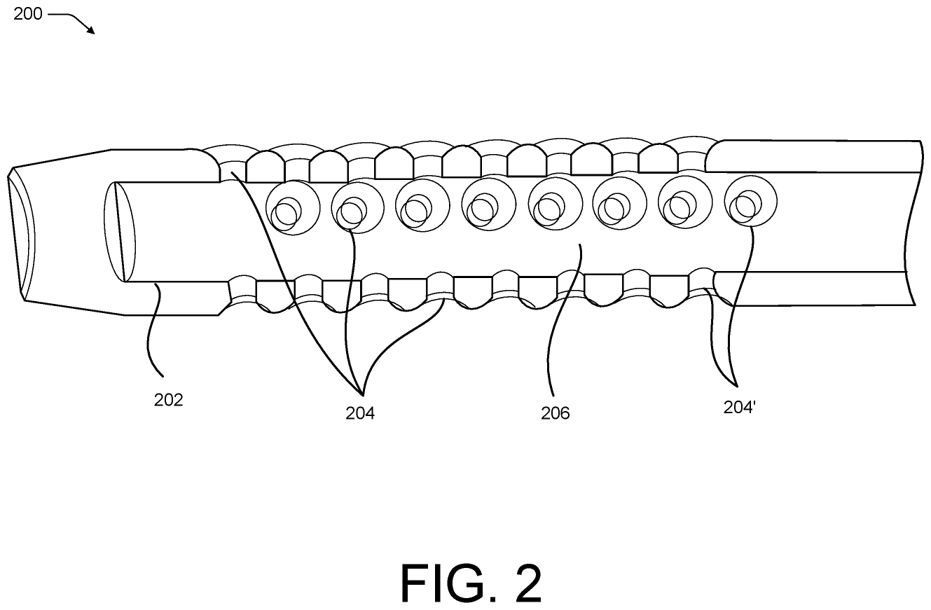

In implementations, hydrocephalus shunts come in various shapes and designs. FIG. 2 illustrates an example conventional catheter 200. Referring to FIG. 2, the catheter 200 includes a tip end 202, multiple holes 204, and a lumen 206. In this example, the catheter 200 is cylindrical, and the radius thereof is the same throughout the catheter 200. The catheter 200 includes multiple holes that allow fluid to flow therethrough into the lumen 204.

Among the multiple holes, the quantity of fluid increases as the fluid flows closer to the holes far from the tip end 202 (such as the holes 204′), which means the velocity of the fluid needs to increase. Increase in fluid velocity results in a reduction in pressure. A pressure gradient is created in the lumen 204 as the result of the flow pattern inside the lumen 204 of the catheter 200. This results in preferential flow through the holes with the highest fluid velocity (the lowest pressure). That is, the holes that are farthest from the tip end 202 (such as the holes 204′) can have more fluid flow therethrough than other holes. As such, the blockage can occur more often around the holes (such as the holes 204′) furthest from the catheter tip.

Therefore, there is a need to reduce the pressure gradient by maintaining a constant low velocity inside the lumen which can result in flow through all the catheter holes and can introduce additional pathways for fluid to go through. This can be done by optimizing the geometry of the catheter.

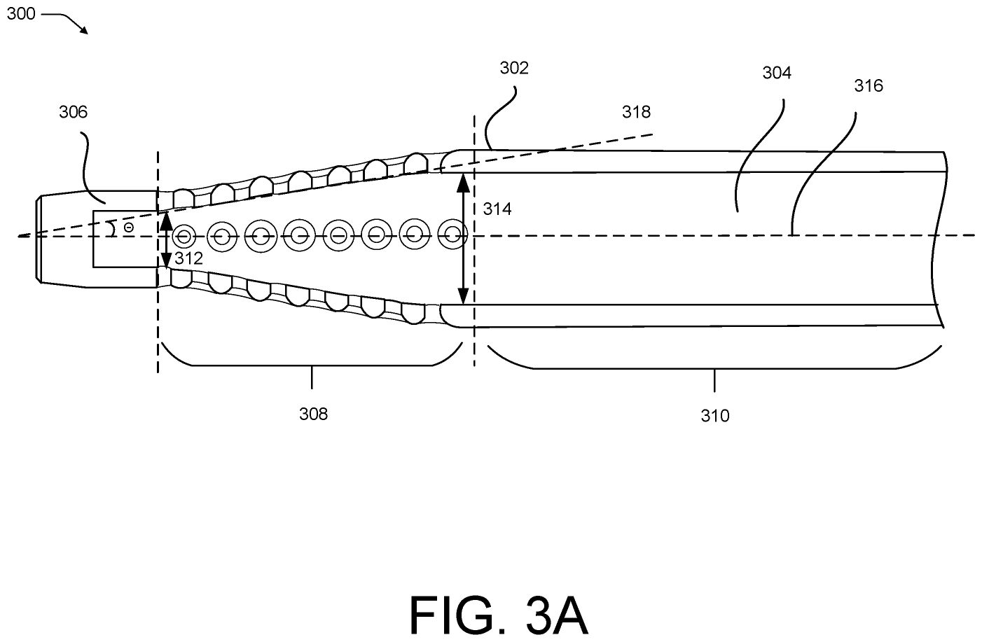

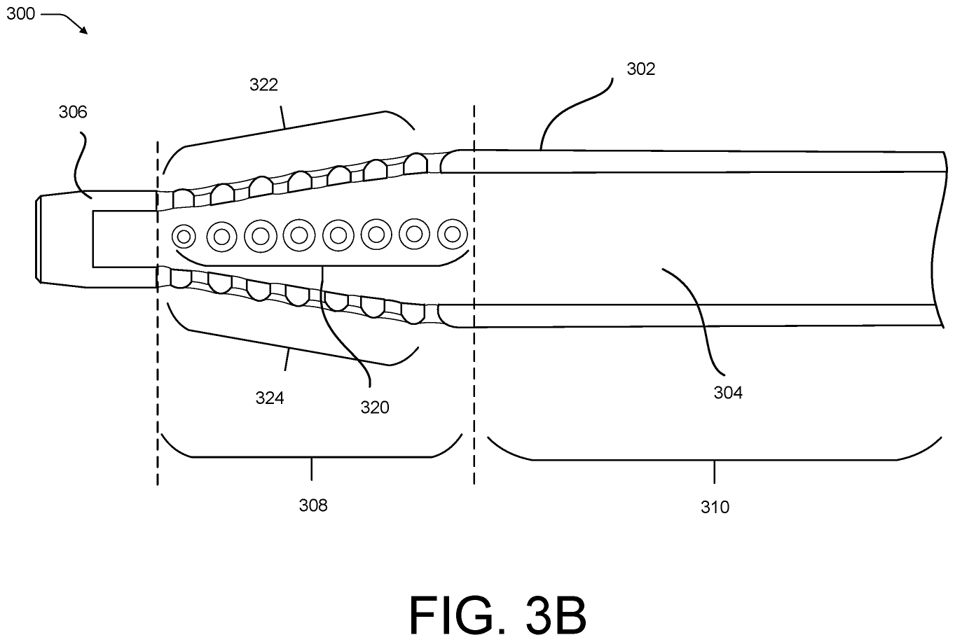

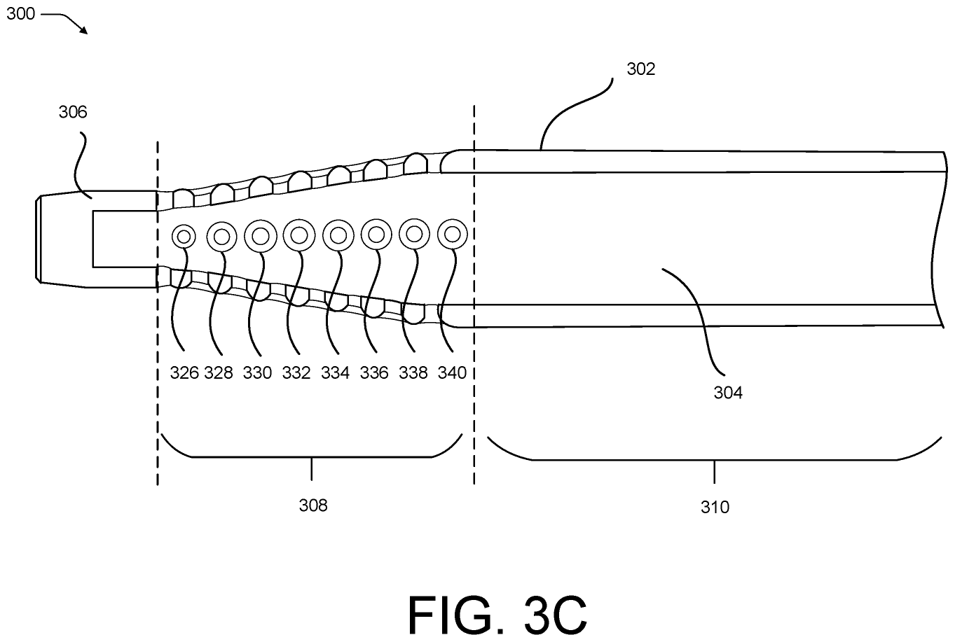

FIG. 3A, FIG. 3B, and FIG. 3C illustrate an example catheter 300 in accordance with this disclosure. As described herein, the catheter 300 can be used for shunting flow of biological fluids in organs or tissues including in vivo or in vitro such as in the context of cerebrospinal fluid in brain tissue, and blood in the circulatory system. For example, the catheter 300 can be implanted into a patient's brain ventricle to shunt CSF to treat hydrocephalous. In some examples, the catheter 300 can be used in vitro to study fluid flow. In some examples, the catheter 300 has an axisymmetric shape such as a cylindrical shape or the like. Moreover, the catheter 300 can have other shapes as long as the shape is suitable for shunting biological fluids.

Referring to FIG. 3A, the catheter 300 includes a sidewall 302. The sidewall 302 defines a lumen 304 of the catheter 300. The catheter 300 has a tip end 306, a first portion 308, and a second portion 310. The tip end 306 is a closed end. The first portion 308 is between and in fluid communication with the tip end 306 and the second portion 310. The first portion 308 can be configured to have a tapered shape, meaning that the diameter of the lumen 304 of the catheter expands gradually or step-wise from the tip end 306 toward the second portion 310. For example, the first portion 308 has a first diameter 312 near the tip end 306, and a second diameter 314 near the second portion 310. In some examples, the first diameter 312 is smaller than the second diameter. In some examples, the first diameter 312 is between 1 millimeter (mm) and 4 mm. The second diameter 314 is between 2 mm and 5 mm. Examples of the first diameter 312 can be 1.5 mm, 1.6 mm, 1.7 mm, 3.5 mm, or the like. Examples of the second diameter 314 can be 2.45 mm, 2.8 mm, 3 mm, 4.5 mm, or the like

As shown in FIG. 3A, the catheter 300 can have a central axis 316. The central axis 316 is a hypothetical line that passes through the center of the catheter longitudinally. In the first portion 308, the sidewall 302 can have a slope 318 towards the tip end 306. An angle θ of the slope 318 with respect to the central axis 316 can be between 0 degree and 30 degrees. The second portion 310 has a constant diameter such as the second diameter 314.

In some examples, the ratio of the first diameter 312 to the second diameter 314 can be between 1:3 and 3:5. In some examples, instead of having a linear sloped sidewall, the first portion 308 can have a curved sidewall. In some examples, the first portion 308 can have a step-wise sidewall.

Referring to FIG. 3B, the catheter 300 can include one or more rows of holes on the sidewall 302 that allow biological fluid flow therethrough from an outside environment (such as a brain ventricle or the like) to the lumen 304 of the catheter 300. For example, in the first portion 308, the sidewall 302 includes a first row of holes 320, a second row of holes 322, a third row of holes 324, and a fourth row of holes (on the backside of the sidewall, not shown). In some examples, the shape of an individual hole can be round, square, or any other suitable shapes.

As an example, referring to FIG. 3C, the first row of holes 320 includes a first hole 326, a second hole 328, a third hole 330, a fourth hole 332, a fifth hole 334, a sixth hole 336, a seventh hole 338, and an eighth hole 340. Note that the number of holes is exemplary rather than limiting, and there can be other numbers of holes. In some examples, the first hole 326, the second hole 328, the third hole 330, the fourth hole 332, the fifth hole 334, the sixth hole 336, the seventh hole 338, and an eighth hole 340 can have the same diameter. In some examples, the first hole 326, the second hole 328, the third hole 330, the fourth hole 332, the fifth hole 334, the sixth hole 336, the seventh hole 338, and an eighth hole 340 can have different diameters. In some examples, the diameter of an individual hole can be between 200 μm and 700 μm. Examples of the diameter of an individual hole can include 600 μm, 750 μm, 450 μm, 460 μm, 275 μm, or the like.

In some examples, the first hole 326, the second hole 328, the third hole 330, the fourth hole 332, the fifth hole 334, the sixth hole 336, the seventh hole 338, and an eighth hole 340 can be placed with constant intervals. In some examples, the first hole 326, the second hole 328, the third hole 330, the fourth hole 332, the fifth hole 334, the sixth hole 336, the seventh hole 338, and an eighth hole 340 can be placed with various intervals. In some examples, the intervals between the holes can be between 0.5 mm to 4 mm. Examples of the intervals can include 3.7 mm, 3.45 mm, 1.1 mm, 0.8 mm, or the like.

Though FIG. 3A, FIG. 3B, and FIG. 3C shows that the multiple rows of holes are arranged in a linear manner, it should be understood that, the holes can be arranged in other manners. In some examples, the multiple rows of holes can be arranged in a spiral manner. In some examples, the holes can be arranged in a staggered manner.

### (II) Shunt Systems Comprising the Catheter

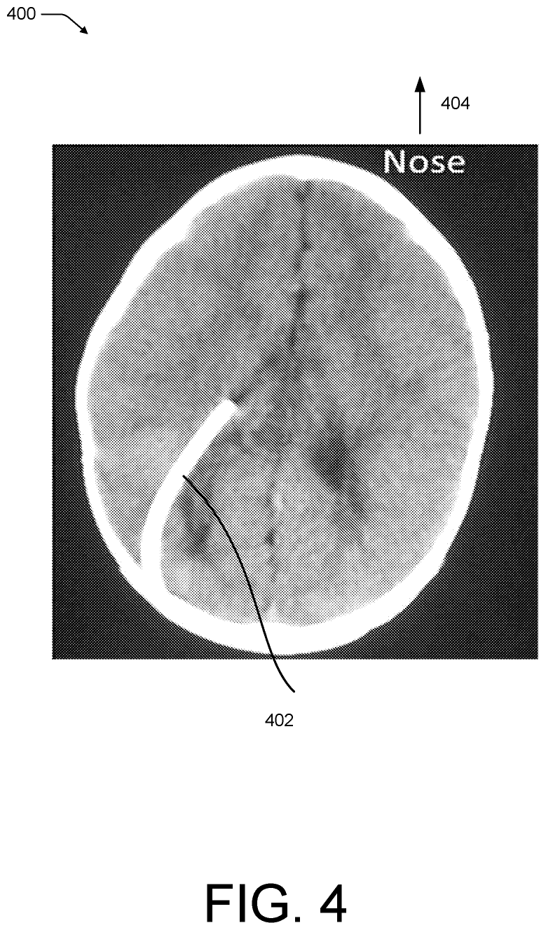

As described herein, the common treatment for hydrocephalus patients is CSF drainage by shunting. A shunt system can be implemented into a patient's brain by surgical insertion to treat hydrocephalous patients. FIG. 4 illustrates a horizontal head CT image 400 showing a catheter tip implanted in a patient's brain. Arrow 404 shows the direction of the patient's nose. Referring to FIG. 4, the image 400 shows a catheter tip 402 surrounded by tissue. While many factors such as infection and disconnection could lead to shunt obstruction and eventual failure, the statistics tell us that most shunts fail by becoming blocked with cells and tissues. As described above, shunt geometry with a more uniform flow rate distribution among the shunt's inlet holes would reduce the obstruction occurring at the critical proximal inlet holes, thereby reducing shunt failure rates.

This disclosure further provides a shunt system comprising the catheter for shunting biological fluid flow as described above. Such a shunt system offers a more uniform flow rate distribution among the inlet holes of the catheter, and would reduce the obstruction occurring at the inlet holes, thereby reducing shunt failure rates. The shunt system is can improve patients' quality of life by reducing the shunt failure rate. In some instances, the system can also be used to conduct in vitro experiments, collect analytic data, validate shunt functions, etc.

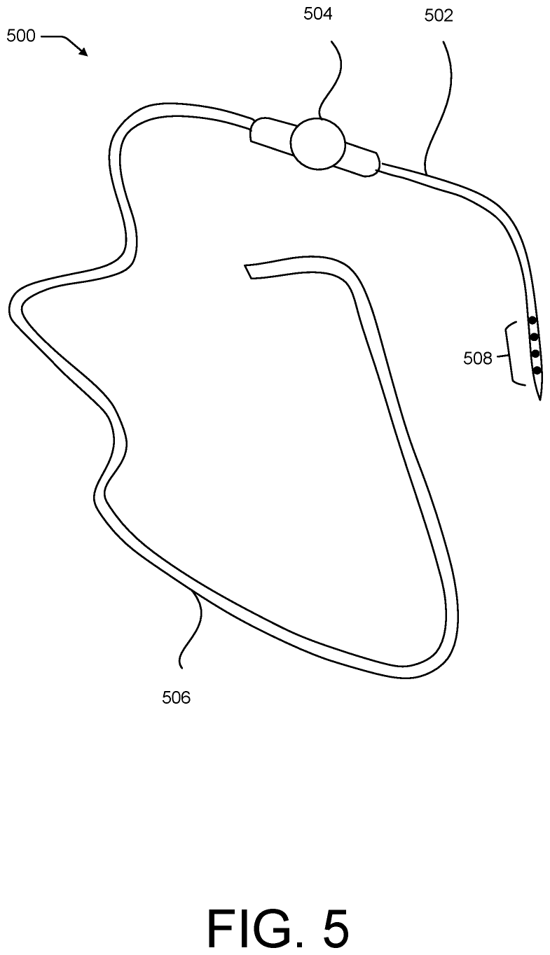

FIG. 5 illustrates an example shunt system 500 in accordance with implementations of this disclosure. In some examples, the shunt system 500 can be used in vitro to study fluid flow, or in vivo to treat or ameliorate symptoms of hydrocephalus. Referring to FIG. 5, the shunt system 500 includes a ventricle catheter 502, a valve 504, and a distal catheter 506. The valve 504 is connected between the ventricular catheter 502 and the distal catheter 506. In some examples, the shunt system 500 can be made of biologically compatible material such as polydimethylsiloxane (PDMS, silicone) or the like.

The ventricle catheter 502 can be implemented into a patient's brain ventricle. The ventricle catheter 502 includes multiple holes 508 that allow biological fluid flow through into the ventricle catheter 502. The ventricle catheter 502 can be configured in the same way as the catheter 300 described with respect to FIG. 3A, FIG. 3B, and FIG. 3C.

The valve 504 is configured to regulate the biological fluid (such as CSF) flowing therethrough. In some examples, the valve 504 can be opened and closed. In some examples, the valve 504 can regulate the flow rate of the biological fluid. In some examples, the valve 504 can be a conventional valve. In some examples, the valve 504 can be a solid state valve described in a sister PCT application (PCT Application No. PCT/US2022/076230, filed Sep. 9, 2022).

The distal catheter 506 is configured to introduce the biological fluid to another part of the body, such as the abdomen through the peritoneum of the patient. As such, excess CSF of the hydrocephalous patient can be drained from the brain to another part of the body where CSF can be more easily absorbed.

Example shunts are composed of two polydimethylsiloxane (PDMS, silicone) shunt catheters connected by a pressure valve. One catheter remains in the ventricles, while the other is tunneled subcutaneously into the peritoneum or atrium.

### (III) Kits

Also provided are kits useful for treating hydrocephalus patients. An example of the kit includes one or more of: a ventricular catheter, a shunt valve, and a distal catheter, each of which is sterile, and vacuum sealed. The ventricular catheter can be configured in the same way as the catheter 300 described with respect to FIG. 3A, FIG. 3B, and FIG. 3C.

More generally, kits can include instructions, for example, written instructions, on how to use the material(s) therein. Material(s) can be, for example, any substance, composition, solution, etc., herein or in any patent, patent application publication, reference, or article that is incorporated by reference.

A kit can include a shunt system as described herein, and optionally additional components such as buffers, reagents, and instructions for carrying out the methods described herein. The choice of buffers and reagents will depend on the particular application, e.g., setting of the assay (point-of-care, research, clinical), analyte(s) to be assayed, the detection moiety used, the detection system used, etc.

The kit can also include informational material, which can be descriptive, instructional, marketing, or other material that relates to the methods described herein and/or the use of the devices for the methods described herein. In embodiments, the informational material can include information about the production of the device, physical properties of the device, date of expiration, batch or production site information, and so forth.

The Example(s) and Example Clauses below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (IV) EXAMPLE(S)

### Example 1

As described herein, catheters and systems including the same were developed to reduce the pressure gradient by maintaining a constant low velocity inside the catheter. Such a system could facilitate flow through all the catheter holes and introduce additional pathways for fluid to go through. The shunt geometry was designed to facilitate a more uniform flow rate distribution among the shunt's inlet holes which would reduce the obstruction at the inlet holes, thereby reducing shunt failure rates. To validate whether the techniques described herein could work as expected, simulations were conducted, and data were collected.

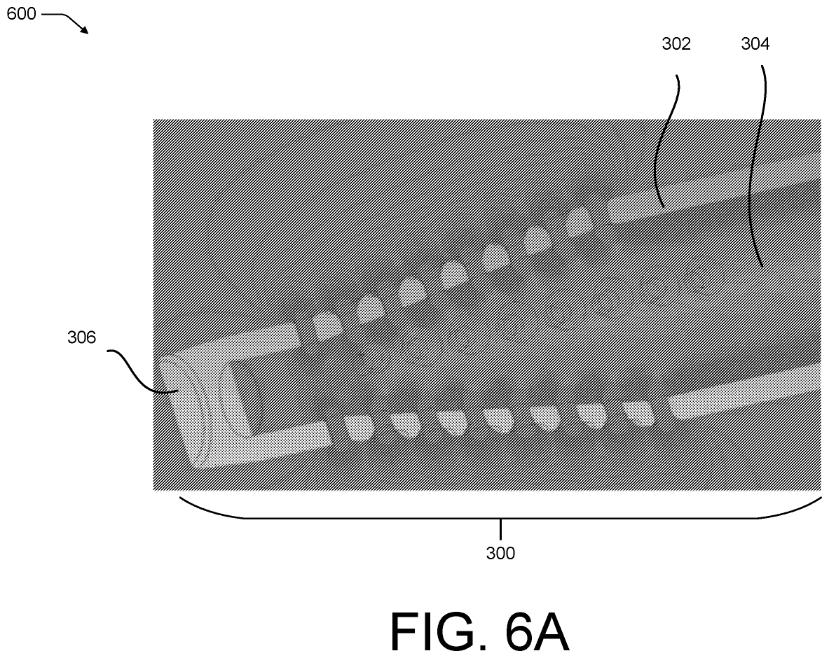

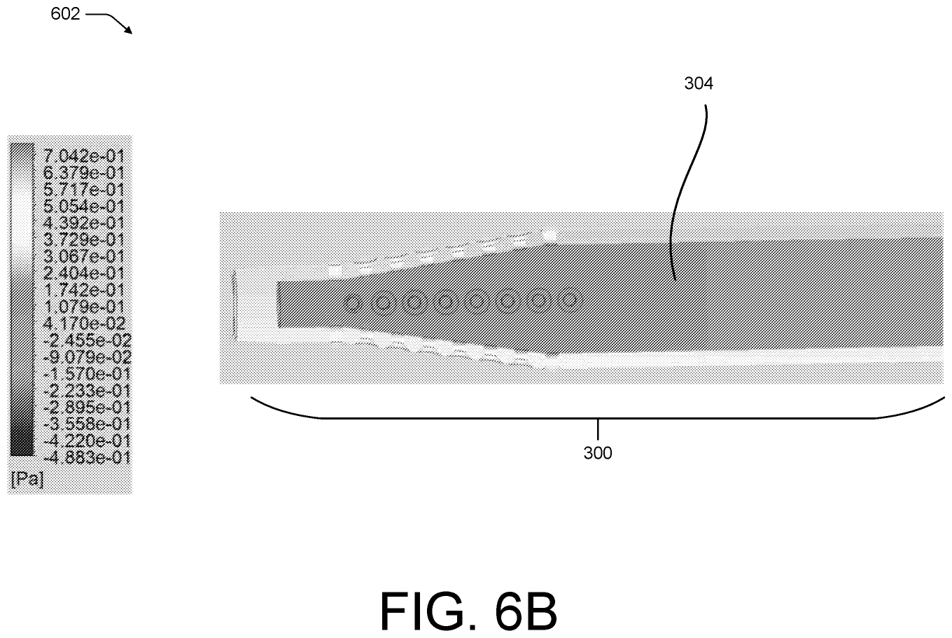

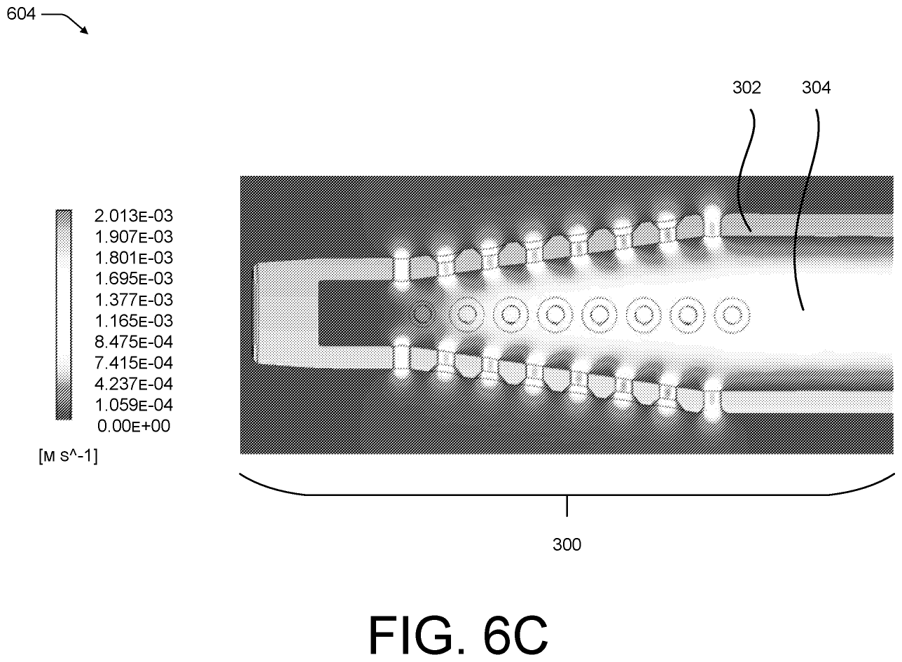

FIG. 6A shows a simulation result 600 of the hydrodynamics of flow through the catheter 300 in accordance with implementations of this disclosure. In FIG. 6A, the greyscale indicated the velocity of the fluid. The darker the color was, the lower the velocity was. Note that the simulation result was schematic rather than limiting. There can be other manners to analyze the characteristic of the catheter 300. Referring to FIG. 6A, the velocity in the lumen 304 of the catheter 300 was substantially constant except for areas around the sidewall 302. As a result, the pressure gradient inside the lumen 304 were reduced by maintaining a substantially constant low velocity inside the lumen 304. Moreover, the fluid flew through most of the catheter holes rather than a few catheter holes that are farthest from the tip end 306. Therefore, it can be seen that the catheter 300 achieved a more uniform flow rate distribution among the shunt's inlet holes which would reduce the obstruction at the inlet holes. As such, shunt failure rates can be reduced.

FIG. 6B illustrates a simulation result 602 of the pressure contours inside the catheter 300 in accordance with implementations of this disclosure. Referring to FIG. 6B, the pressure in the lumen 304 of the catheter 300 was substantially constant. In other words, the pressure gradient inside the lumen 304 decreased.

FIG. 6C illustrates a simulation result 604 of the velocity contours inside the catheter 300 in accordance with implementations of this disclosure. Referring to FIG. 6C, the velocity in the lumen 304 of the catheter 300 was substantially constant except for areas around the sidewall 302. Moreover, the fluid flew through most of the catheter holes rather than a few catheter holes. Therefore, it can be seen that the catheter 300 achieved a more velocity distribution among the inlet holes which would reduce the obstruction at the inlet holes. As such, shunt failure rates can be reduced.

In some examples, the simulations were conducted using commercial software such as ANSYS, COMSOL, or the like.

### (V) Example Clauses

A: A catheter for shunting fluid, comprising a tip end, a first portion, and a second portion; wherein the first portion is between and in fluid communication with the tip end and the second portion; the first portion is configured to have a tapered shape; and the first portion comprises multiple rows of holes.
B: The catheter of clause A, wherein the tip end is a closed end.
C: The catheter of either clause A or B, wherein the first portion has a first diameter close to the tip end and a second diameter close to the second portion, the first diameter being smaller than the second diameter.
D: The catheter of clause C, wherein the second portion has a constant diameter.
E: The catheter of either clause C or D, wherein the second portion has substantially the same diameter as the second diameter of the first portion.
F: The catheter of any one of clauses A-E, wherein an individual row of the multiple rows of holes comprises multiple holes, the multiple holes being placed at constant intervals.
G: The catheter of any one of clauses A-F, wherein an individual row of the multiple rows of holes comprises multiple holes, the multiple holes being placed at various intervals.
H: The catheter of any one of clauses A-G, wherein an individual row of the multiple rows of holes comprises multiple holes, the multiple holes having the same diameter.
I: The catheter of any one of clauses A-H, wherein an individual row of the multiple rows of holes comprises multiple holes, the multiple holes having different diameters.
J: The catheter of any one of clauses A-I, wherein an individual row of the multiple rows of holes comprises multiple holes, the multiple holes are configured to facilitate a relatively uniform flow rate distribution of the fluid flowing therethrough.
K: A system for shunting fluid, comprising: a ventricular catheter, comprising a tip end, a first portion, and a second portion; wherein the first portion is between and in fluid communication with the tip end and the second portion; the first portion is configured to have a tapered shape; and the first portion comprises multiple rows of holes; a distal catheter; and a valve connected between the ventricular catheter and the distal catheter.
L: The system of clause K, wherein the tip end is a closed end.
M: The system of either clause K or L, wherein the first portion has a first diameter close to the tip end and a second diameter close to the second portion, the first diameter being smaller than the second diameter.
N: The system of any one of clauses K-M, wherein the second portion has a constant diameter.
O: The system of any one of clauses K-N, wherein the second portion has a second diameter.
P: The system of any one of clauses K-O, wherein an individual row of the multiple rows of holes comprises multiple holes, the multiple holes being placed at constant intervals.
Q: The system of any one of clauses K-P, wherein an individual row of the multiple rows of holes comprises multiple holes, the multiple holes being placed at various intervals.
R: The system of any one of clauses K-Q, wherein an individual row of the multiple rows of holes comprises multiple holes, the multiple holes having the same diameter.
S: The system of any one of clauses K-R, wherein an individual row of the multiple rows of holes comprises multiple holes, the multiple holes having different diameters.
T: The system of any one of clauses K-S, wherein an individual row of the multiple rows of holes comprises multiple holes, the multiple holes are configured to facilitate a relatively uniform flow rate distribution of the fluid flowing therethrough.
U: A ventricular catheter essentially as described or illustrated herein.
V: The ventricular catheter of clause U, which is optimized to reduce astrocyte activation.
W: The ventricular catheter of clause V, wherein the optimization comprises at least one of shear reduction or flow redistribution.
X: The ventricular catheter of any one of any one of clauses U-W, formatted for use with a hydrocephalus shunt.
Y: Use of the ventricular catheter of any one of clauses U-W in vitro to study fluid flow, or in vivo to treat or ameliorate symptoms of hydrocephalus.

While the example clauses described above are described with respect to one particular implementation, it should be understood that, in the context of this document, the content of the example clauses can also be implemented via a method, device, system, and/or a computer-readable medium.

### (VII) REFERENCES

Gluski et al., Fluids Barriers CNS 17(1):46, 2020 (doi: 10.1186/s12987-020-00211-6)
Horbatiuk et al., RSC Adv. 10(52):31056-30164, 2020 (doi: 10.1039/d0ra05128d)
Khodadadei et al., Commun. Biol. 4(1):387, 2021 (doi: 10.1038/s42003-021-01888-7)
Harris et al., Fluids Barriers CNS 18(1):4, 2021 (doi: 10.1186/s12987-021-00237-4)

### (VIII) Closing Paragraphs

Specific descriptions provided herein and in the herewith filed documents are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, references have been made to patents, printed publications, journal articles, and other written text throughout this specification (referenced materials herein). Each of the referenced materials is individually incorporated herein by reference in their entirety for their referenced teaching.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

1. A catheter for shunting fluid, comprising a tip end, a first portion, and a second portion; wherein
the first portion is between and in fluid communication with the tip end and the second portion;
the tip end is configured to have a cylindrical shape;
the first portion is configured to have a tapered shape with a diameter expanding from a first diameter adjacent the tip end to a second, larger diameter adjacent the second portion; and
the first portion comprises multiple rows of holes.

2. The catheter of claim 1, wherein the tip end is a closed end.

3. (canceled)

4. The catheter of claim 1, wherein the second portion has a constant diameter.

5. The catheter of claim 1, wherein the second portion has substantially the same diameter as the second diameter of the first portion.

6. The catheter of claim 1, wherein an individual row of the multiple rows of holes comprises multiple holes, the multiple holes being placed at constant intervals.

7. The catheter of claim 1, wherein an individual row of the multiple rows of holes comprises multiple holes, the multiple holes being placed at various intervals.

8. The catheter of claim 1, wherein an individual row of the multiple rows of holes comprises multiple holes, the multiple holes having the same diameter.

9. The catheter of claim 1, wherein an individual row of the multiple rows of holes comprises multiple holes, the multiple holes having different diameters.

10. The catheter of claim 1, wherein an individual row of the multiple rows of holes comprises multiple holes, the multiple holes are configured to facilitate a relatively uniform flow rate distribution of the fluid flowing therethrough.

11. A system for shunting fluid, comprising:
a ventricular catheter, comprising:
a uniformly cylindrical tip end, a first portion, and a second portion; wherein
the first portion is between and in fluid communication with the tip end and the second portion;
the first portion is configured to have a tapered shape; and
the first portion comprises multiple rows of holes;
a distal catheter; and
a valve connected between the ventricular catheter and the distal catheter.

12. The system of claim 11, wherein the tip end is a closed end.

13. The system of claim 11, wherein the first portion has a first diameter close to the tip end and a second diameter close to the second portion, the first diameter being smaller than the second diameter.

14. The system of claim 11, wherein the second portion has a constant diameter.

15. The system of claim 11, wherein the second portion has a second diameter.

16. The system of claim 11, wherein an individual row of the multiple rows of holes comprises multiple holes, the multiple holes being placed at constant intervals.

17. The system of claim 11, wherein an individual row of the multiple rows of holes comprises multiple holes, the multiple holes being placed at various intervals.

18. The system of claim 11, wherein an individual row of the multiple rows of holes comprises multiple holes, the multiple holes having the same diameter.

19. The system of claim 11, wherein an individual row of the multiple rows of holes comprises multiple holes, the multiple holes having different diameters.

20. The system of claim 11, wherein an individual row of the multiple rows of holes comprises multiple holes, the multiple holes are configured to facilitate a relatively uniform flow rate distribution of the fluid flowing therethrough.

21.-25. (canceled)

26. The system of claim 11, the ventricular catheter further comprising a lumen extending from the tip portion through the first portion and the second portion, wherein the ventricular catheter is configured to maintain a constant low velocity inside the lumen when implanted in a human.
